(19) United States
(12) Patent Application Publication
(10) Pub. No.: US 2009/0181438 A1
(43) Pub. Date: Jul. 16, 2009

(54) OPTIMIZATION OF BIOFUEL PRODUCTION

(75) Inventor: Richard T. Sayre, Webster Groves, MO (US)

Correspondence Address: STANDLEY LAW GROUP LLP, 6300 Riverside Drive, Dublin, OH, 43017, US

(73) Assignee: THE OHIO STATE UNIVERSITY RESEARCH FOUNDATION, Columbus, OH (US)

(21) Appl. No.: 12/328,695

(22) Filed: Dec. 4, 2008

Related U.S. Application Data
(60) Provisional application No. 60/992,261, filed on Dec. 4, 2007.

(51) Int. Cl. C12P 7/64; C12M 1/00

(52) U.S. Cl. 435/134; 435/289.1

(57) ABSTRACT

Embodiments of the present invention includes an apparatuses, compositions, and methods utilizing mechanical and chemical engineering strategies to achieve even greater efficiencies in biofuels production from oleaginous organisms. These increased efficiencies may be achieved through the application of targeted and well-designed chemical and mechanical engineering methods disclosed herein to achieve a non-destructive extraction process (NDEP).

### CROSS-REFERENCE TO RELATED APPLICATIONS

This non-provisional patent application claims the benefit of priority from U.S. Provisional Patent Application No. 60/992,261 filed Dec. 4, 2007, which is hereby incorporated by reference in its entirety.

### TECHNICAL FIELD

The disclosed embodiments of the present invention are in the field of systems and methods for biofuel production, particularly systems and methods of producing biofuels that utilize microalgae.

### BACKGROUND

Recently, the price of petroleum has fluctuated dramatically, reaching record highs as well as making dramatic downwards swings. In part, the recent price increases reflect political and supply chain uncertainties. Concern about the availability of inexpensive petroleum supplies has lead to the growing realization that energy independence for an industrialized nation is of critical strategic importance. There also is general agreement now that the release of CO₂ from fossil fuel combustion has contributed substantially to global warming and climate change. As a result of these concerns, the domestic production of carbon neutral biofuels has become an increasingly attractive alternative to the consumption of imported fossil fuels.

Between the late 1970s and 1990s, the US Department of Energy's National Renewable Energy Labs (NREL) evaluated the economic feasibility of producing biofuels from a variety of aquatic and terrestrial photosynthetic organisms (Sheehan et al., 1998). Biofuel production from microalgae was determined to have the greatest yield/acre potential of any of the organisms screened. Microalgal biofuel production was estimated to be 8 to 24 fold greater than the best terrestrial biofuel production systems. Although promising, there is still a need for compositions, systems, and methods that provide even greater efficiencies in biofuel production from microalgae.

### SUMMARY OF THE INVENTION

This and other unmet needs of the prior art are met by exemplary compositions, systems, and methods described in more detail below.

In one aspect, embodiments of the present invention utilize mechanical and chemical engineering strategies to achieve even greater efficiencies in biofuels production from oleaginous organisms. These increased efficiencies may be achieved through the application of targeted and well-designed chemical and mechanical engineering methods disclosed herein to achieve a non-destructive extraction process (NDEP).

Accordingly, provided herein is a method for oil extraction from an oleaginous organism, comprising:

a mixing step, which includes mixing at least a portion of a culture containing an oleaginous organism with a solvent that extracts oil from the oleaginous organism to obtain a solvent-organism mixture;

an extraction step, which includes directing the solvent-organism mixture into a partitioning chamber to obtain an extracted aqueous fraction containing a viable extracted organism and a solvent-oil fraction; and

a recycling step, in which at least a portion of the viable extracted organism is recycled into a culturing system.

In one embodiment, the method further comprises the step of distilling the solvent-oil fraction to obtain a usable oil.

In another embodiment. the method further comprises the steps of: distilling the solvent-oil fraction to obtain a usable oil and recovered solvent; and recycling at least a portion of the recovered solvent for use in the mixing step.

In another embodiment, the method is performed so that the oleaginous organism undergoes at least two separate cycles of mixing and oil extraction.

In some embodiments, the oleaginous organism is an alga.

In other embodiments, the oleaginous organism is an oleaginous yeast.

In yet other embodiments, the oleaginous organism is an oleaginous fungus.

In some embodiment, the solvent used in the method includes one or more of C4-C16 hydrocarbons. In some embodiments, the solvent includes a C10, C11, C12, C13, C14, C15, or C16 hydrocarbon. In one embodiment, the solvent is Isopar.

The oleaginous organism used in the method may be genetically engineered to enhance lipid production.

In some embodiments, the oleaginous organism is concentrated prior to oil extraction.

In some examples, sonication is used during at least a portion of the mixing step. The sonication can be performed at a frequency between about 20 kHz and 1 MHz, 20-100 kHz, 20-60 Khz, 30-50 Khz, or at 40 Khz. In alternative embodiments, the mixing step may be facilitated instead with the use of mechanical mixing (e.g., agitation). In still other embodiments, sonication and mechanical mixing may be used in combination.

In another aspect, provided herein is a method for oil extraction from an oleaginous alga, comprising: mixing at least a portion of a culture containing the alga with a solvent that extracts oil from the alga to obtain a solvent-alga mixture; directing the solvent-alga mixture into a partitioning chamber to obtain an extracted aqueous fraction containing a viable extracted alga and a solvent-oil fraction; and recirculating at least a portion of the viable extracted alga into a culturing system.

Also provided herein is a method for oil extraction from a photosynthetic oleaginous organism, comprising: mixing at least a portion of a culture containing the photosynthetic oleaginous organism with a solvent that extracts oil from the organism to obtain a solvent-organism mixture; directing the solvent-organism mixture into a partitioning chamber to obtain an extracted aqueous fraction containing a viable extracted organism and a solvent-oil fraction; and recirculating a portion of the viable extracted organism into a culturing system.

In some embodiments, the method further comprises the step of: providing a wavelength-shifting dye, the dye adapted to increase the quantity of usable photons available to the photosynthetic alga in the culture system. The wavelength-shifting dye can be incorporated into particles, or into a film.

In some embodiments, the method further comprises the step of: providing a Fresnel lens adapted to increase the quantity of photons available to the photosynthetic alga when a light source is received at oblique angles.

In some embodiments, the method further comprises the step of: distilling the solvent-oil fraction to obtain a usable oil.

All the methods and processes disclosed herein may be performed in a continuous fashion.

In some embodiments, an apparatus is included for carrying out the disclosed method.

Exemplary embodiments of the compositions, systems, and methods disclosed herein may be used individually or in various combinations to enhance lipid production and oil extraction from microalgae. Embodiments disclosed herein may enhance lipid production by increasing solar energy utilization efficiency, cell culture density, and using novel lipid harvesting technologies to non-destructively harvest oils from live cultures.

### BRIEF DESCRIPTION OF THE DRAWINGS

A better understanding of the exemplary embodiments of the invention will be had when reference is made to the accompanying drawings, and wherein:

FIG. 1 is a graph showing the effects of alkane solvent treatment on the survivability of Chlorella protothecoides cells.

FIG. 2 is a graph showing the effects of alkane solvent treatment with or without sonication on the extraction of lipids (total fatty acids (FA)) from live cells.

FIG. 3 schematically shows an exemplary device which may be used for the non-destructive extraction of oil from algae.

FIG. 4 is a diagram of an exemplary system and method for the non-destructive extraction of oil from algae.

FIG. 5 includes data showing the effect of different levels of sonication coupled with decane extraction on viability of the green alga Chlorella protothecoides.

FIG. 6 is a plot demonstrating that solvent extractions can be performed daily to recover more oil or neutral lipids.

FIG. 7 Repetitive solvent extraction yields more oil. Summary of total biomass and non-destructively extracted neutral lipids of daily versus batch (3^{rd} day only) extracted cultures.

FIG. 8 shows growth of Nannochloropsis is not impaired after multiple cycles of non-destructive lipid extraction.

FIG. 9 demonstrates effects of solvent (decane) exposure coupled with sonication on the viability of Nannochloropsis sp.

FIG. 10 is a plot demonstrating growth of Nannochloropsis sp. under different non-destructive extraction processes.

FIG. 11 is a plot showing the differing growth rates of Nannochloropsis sp. after extraction with various solvents facilitated by a sonication step.

FIG. 12 Design of transforming plasmids tested for reduction of chlorophyll b and the light harvesting complex. The plasmids either overexpress chlorophyll b reductase, which would convert chlorophyll b back to chlorophyll a (plasmid 1), or are RNAi constructs to reduce the activity of chlorophyll a oxidase (CAO, plasmids 2-5), which synthesizes chlorophyll b from chlorophyll a.

FIG. 13 Transformation frequency and changes in chlorophyll a/b ratios in transgenic organism showing a reduction in chlorophyll b content.

FIG. 14 is an explanation of chlorophyll kinetic analysis of light harvesting complex contributions to the rise and decay of chlorophyll fluorescence.

FIG. 15 shows transgenic algae with slower chlorophyll fluorescence rise kinetics and lower maximum chlorophyll fluorescence levels consistent with a reduction in light harvesting complex. Transformants were made using plasmid construct 4 in FIG. 12 which would reduce expression of chlorophyll a oxidase, the enzyme that makes chlorophyll b from chlorophyll a.

FIG. 16 is a table showing the factors limiting photosynthetic efficiency.

FIG. 17 is a diagram demonstrating that a major window of visible light ranging between 400 and 600 nm is not absorbed efficiently by chlorophyll.

FIG. 18 shows a series of exemplary dyes that may be useful for increasing the number of photons harvestable by the photosynthetic machinery.

FIG. 19 illustrates one of the techniques that may be useful for increasing light capture. The benefits of a Fresnel lens are shown here schematically.

### DETAILED DESCRIPTION

Unless otherwise defined, all technical and scientific terms used herein have the same meaning as commonly understood by one of ordinary skill in the art to which this invention pertains. Although methods and materials similar or equivalent to those described herein can be used in the practice or testing of the exemplary embodiments, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein “milking” and “non-destructive extraction” are used to describe a process wherein the organism is treated with a solvent to remove lipids without causing significant loss of viability of the culture. Non-destructive extraction or extraction “essentially without killing” the organism, refers to cycles of extraction and recycling/recirculating of live extracted organisms to the culture system for regrowth or additional lipid and biomass production, and to the concept that the organism will survive at least one extraction cycle, but may be destroyed upon subsequent extraction cycles.

A “culture system” refers broadly to any system useful for culturing an organism. These can be ponds, raceways, bioreactors, plastic bags, tubes, fermentors, shake flasks, air lift columns, and the like.

A “usable oil” refers to oil that is suitable for the production of biofuels. Such oil may or may not be completely free of solvent or other coextractants from the organism.

As used herein a “continuous” extraction process is one in which the mixing/extracting/recycling steps occur continuously with minimal operator input for an extended period but is contemplated to be run and stopped at intervals as needed for maintenance or to maximize extraction productivity.

A “biocompatible” solvent is a solvent that may be contacted to an organism and tolerated by the organism without significant loss in viability. A biocompatible solvents will generally have an octanol number (“log Poct”, the logarithm of the octanol-water partition coefficient) greater than 5. See Frenz J, Largeau C, Casadevall E, Kollerup F, Daugulis A J (1988) Hydrocarbon recovery and biocompatibility of solvents for extraction of cultures of Botryococcus braunii. Biotech Bioeng 34: 755-762. Generally, the log P value correlates well with solvent biocompatibility in that solvents with log Po less than 4 are toxic and solvents with log Po greater than 5 are biocompatible (Dodecmone is one exception to this rule). Solvents with a log Po in the range 4-5 may be toxic (decanol, dipentyl ether) or nontoxic (hexane, heptane) so that no absolute cutoff can be established based solely on this parameter. In part this may reflect some inaccuracies in the calculation of log Po and more accurate values for such solvents may be expected to better correlate with biocompatibility. Exemplary solvents include: 1,12-dodecanedioic acid diethyl ether, n-hexane, n-heptane, n-octane, n-dodecane, dodecyl acetate, decane, dihexyl ether, isopar, 1-dodecanol, 1-octanol, butyoxyethoxyehteane, 3-octanone, cyclic paraffins, varsol, isoparaffins, branched alkanes, oleyl alcohol, dihecylether, 2-dodecane.

The process of “sonication” is the treatment of a sample with high energy sound or acoustical radiation that is referred to herein as “ultrasound” or “ultrasonics.” Sonication is used in the art for various purposes including disrupting aggregates of molecules in order to either separate them or permeabilize them.

Using novel chemical and mechanical engineering strategies, exemplary embodiments of the invention are directed at increasing the yield of energy rich lipids (e.g., triacylglycerol) that may be harvested from algae. Although many of the exemplary embodiments described below may be useful individually, the exemplary compositions, systems, and methods of the current system may work complimentarily to optimize both cost and yield.

The systems and methods disclosed herein may utilize a vast array of oleaginous organisms including alga, yeasts and fungi.

Many algal species may be used with acceptable results. Some alga species include, without limitation: Bacillariophyceae strains, Chlorophyceae, Cyanophyceae, Xanthophyceaei, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thaustochytrids.

Suitable yeasts include, but are not limited to, Rhodotorula, Saccharomyces, and Apiotrichum strains.

Acceptable fungi species include, but are not limted to, the Mortierella strain.

At least one exemplary embodiment utilizes Chlorella protothecoides. C. protothecoides may be especially appropriate because it grows at high culture cell densities, typically 10-fold higher than most algae (Xu et al., 2006; Miao and Wu, 2006). Record biomass yields of up to 35 gfw/L have been recorded for C. protothecoides when grown heterotrophically under ideal conditions. C. protothecoides is capable of accumulating at least 55% of its biomass as lipid, a value that is unmatched by most algal strains. C. protothecoides can be grown heterotrophically on glucose or corn sweetener hydrolysate (CSH). Heterotrophic growth increases lipid content and can reduce direct dependency on solar energy. The energy density of biodiesel produced from C. protothecoides is equivalent to that of petroleum-based diesel (Xu et al., 2006; Miao and Wu, 2006). The cold filter plugging temperature of biodiesel produced from C. protothecoides is lower than that for diesel fuel (Xu et al., 2006; Miao and Wu, 2006). Chlorella as well as other microalgal species have the potential to be genetically engineered and they have been successfully grown in large-scale photobioreactors using flue gasses as sources of enriched CO₂ (Brown, 1996; Doucha and Livansky, 2006; Kadam, 1997; Keffler and Kleinheinz, 2002, Chow and Tung, 1999; Dawson et al., 1997; El-Sheekh, 1999; Chen et al., 2001).

Milking Oils from Algal Cultures without Harming the Algae:

One of the major costs associated with biofuel production is harvesting the biofuel from large volumes of culture media (Becker, 1994). Harvesting, rupturing, drying and extracting oils from algae accounts for 40-60% of the cost of producing biodiesel and places additional demands on culture replenishment. There is a need for a nondestructive, low cost oil extraction technology.

Certain microalgae have a high potential for lipid production. When grown heterotrophically, approximately 15-55% of the cell is lipid. However, even though the lipid content is high, if the lipids cannot be harvested essentially without harming the microalgae, then 45-85% (the non-lipid biomass) of the microalgal biomass will need to be regenerated in order to produce additional useful lipids.

Accordingly, described herein are methods for non-destructive oil extraction from an oleaginous organism, which include: mixing at least a portion of a culture containing an oleaginous organism with a solvent that extracts oil from the oleaginous organism to obtain a solvent-organism mixture; directing the solvent-organism mixture into a partitioning chamber to obtain an extracted aqueous fraction containing a viable extracted organism and a solvent-oil fraction; and a recycling step, in which at least a portion of the viable extracted organism is recycled into a culturing system. In an exemplary system in some ways analogous to a dairy operation, the system allows for the collection of usable oil from the oleaginous organism essentially without rupturing or harming the organism. An embodiment of the extraction process includes solvent extraction and sonication to accomplish “hydrocarbon milking” of the organism. After extraction of the usable oils, the organisms can begin a new process of accumulating lipids. The exemplary processes allows for efficient collection while at the same time preserving the viability of a portion of the cultured organisms. This saves the energy and materials that would otherwise be required to regenerate the live organisms.

Advantageously, the “milking” process may actually benefit the algae. Mixing alkanes with live cultures has also been shown to extend culture growth times from one week to more than five weeks. This effect may be associated with the partitioning of toxic waste products secreted from algae into the hydrophobic fraction of the media (Richmond, 2004).

In the case of algae, the inflation adjusted cost for harvesting cells by centrifugation (biomass=0.1% of the culture volume) is estimated to be $2.40/kg in 2006 (Becker, 1994). Assuming a lipid yield of 55% of the total biomass the cost of centrifugation to produce one gallon of oil from algae is estimated to be $18. Harvesting by flocculation or flotation is only marginally less expensive ($14.60/gallon). Some of these costs can be reduced, however, by growing more dense algal cultures. Assuming a linear relationship between culture density and the cost of harvesting algae, the cost of harvesting algae from cultures having three-fold higher densities (e.g., those lacking LHC complex) would be $4.80/gallon oil produced, still excessively high in today's market where the cost of producing crude oil for gasoline is $1.60/gallon. Harvesting prices would need to drop 3-fold further for biofuel production from algae to be competitive with crude oil production costs.

Recently, it has been demonstrated that very hydrophobic molecules, such as beta-carotene, can be continuously extracted from live algae and bacterial cultures using non-miscible, biocompatible alkanes. These alkanes typically have carbon chain lengths between 10 and 16 atoms (Hejazi et al., 2002; Hejazi and Wijffels, 2004; Hejazi et al., 2004). Continuous mixing of algal cultures with alkanes allows for uninterrupted extraction of beta-carotene. Importantly, the extracted carotenoids come from carotenoid storage vesicles and not chloroplasts. As a result, alkane extraction has no negative impacts on long-term (50 days then stopped) culture growth (Hejazi et al., 2002; Hejazi and Wijffels, 2004; Hejazi et al., 2004).

Some exemplary embodiments disclosed herein utilize “hydrocarbon milking” as a cost-effective means for continuously harvesting oils from algae. In some embodiments, the processes described here do not require centrifugation, have a very high lipid yield, and significantly, the extraction process is essentially harmless (and may even be beneficial) to the algae. Hydrocarbon milking may eliminate the need for centrifugation/flocculation and the destructive solvent (methanol) or mechanical disruption steps typically used to extract oil from algae.

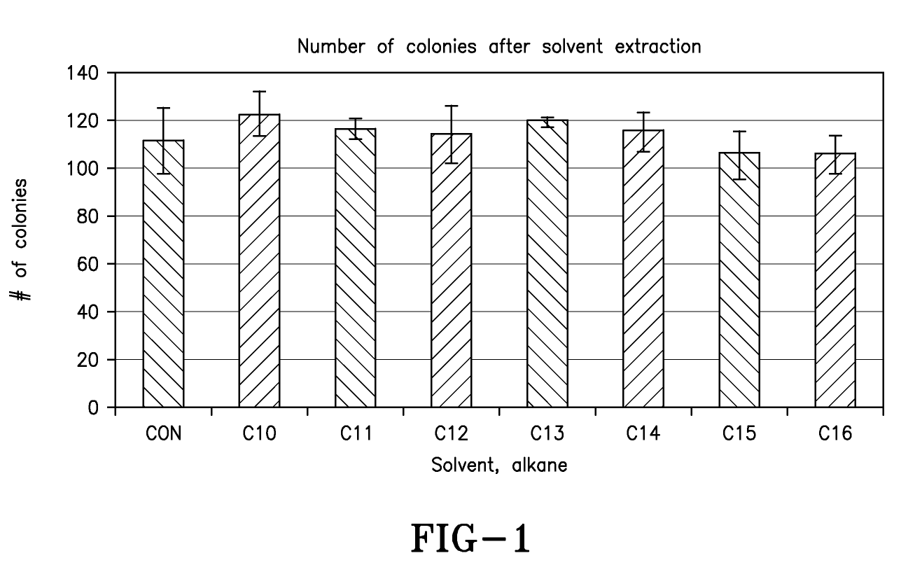

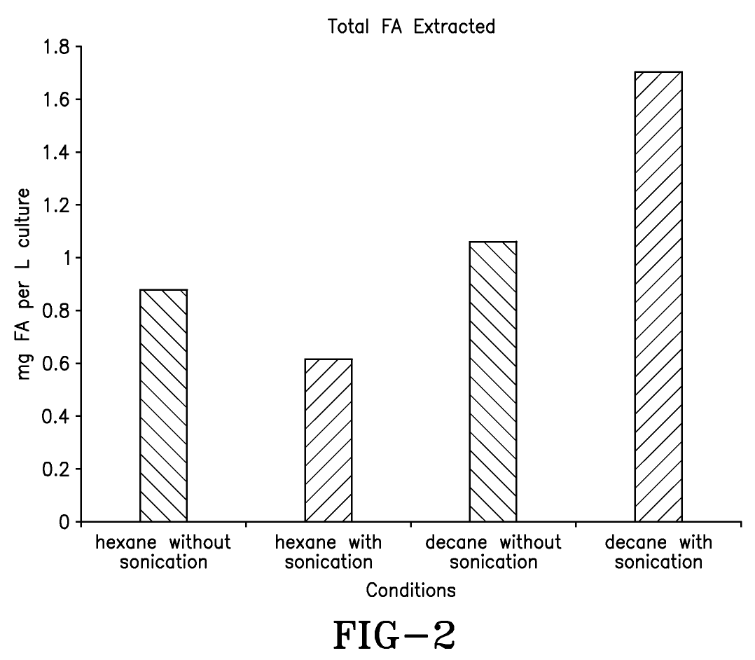

Referring to FIG. 1, in order to determine if lipids may be safely removed from live algal cultures, we extracted air-grown C. protothecoides cultures with hexane, decane and longer chain hydrocarbons and determined whether solvent extraction removed lipids and had an impact on cell viability. Unexpectedly, as shown in FIG. 1, incubation of live cells with C10 to C16 alkanes for 5 minutes had no affect on cell survivability.

Referring to FIG. 2, log phase cultures were treated with various alkanes for 5 minutes plus or minus two seconds sonication. Solvent extracted lipids were saponified and free fatty acids were quantified by LC-MS analysis using C17 internal standards. Significantly, 10% of the total cellular fatty acids were extracted during a five minute exposure to solvents when supplemented with a two second sonication. Importantly, the short sonication enhanced lipid extraction by 75%.

When viewed in concert, FIGS. 1 and 2, results using organic solvents to extract oils from live cells, demonstrate that non-destructive extraction works. Based on an indirect quantification of cellular triacylglycerols using Nile red, nearly 100% of the triacylglycerols present in air-grown cells were extracted by decane during a 5 minute extraction with sonication (FIG. 2). Potentially, short-chain or branched-chain alkanes may also efficiently extract oils from high oil-containing (40% of biomass) algal cells grown in glucose. Solvent extraction time and temperature may be optimized to achieve the most efficient oil extraction from microalgae.

While expressly not limited to theory, sonication is believed to improve oil extraction by breaking up the culture droplets into smaller particles allowing greater solvent exposure to the algae. Ultrasonic irradiation of microorganisms without damaging effects has been shown to be dose dependent at low frequency. As frequency increases, longer irradiation is tolerated by microorganisms (Tiehm, 2001). We use an optimal range of frequencies (20 kHz to 1 MHz) and intensities over different ultrasonic exposure times to optimize the extraction of oils without compromising the viability of cells. However, it should be appreciated that various other frequencies, intensities, and exposure times may also yield acceptable extraction efficiencies.

Exemplary embodiments of the present invention release oils essentially without killing cells. Ultrasonic irradiation of microorganisms without damaging effects has been shown to be dose dependent at low frequency. As frequency increases, longer irradiation is tolerated by microorganisms. An optimal range of frequencies (20 kHz to 60 Khz) and intensities over different ultrasonic exposure times may be utilized to optimize the extraction of oils without compromising the viability of cells. However, it should be appreciated that various other frequencies, intensities, and exposure times may also yield acceptable extraction efficiencies, including frequencies between 20 kHz and 1 MHz, 20-100 kHz, 20-60 Khz, 30-50 Khz, or at 40 Khz. It is known that cell size, cell shape, cell wall composition and physiological state all affect the interaction of ultrasound with cells (Wase and Patel, 1985; Ahmed and Russell, 1975).

In certain embodiments, nearly 100% oil (10% of total fatty acids in cells) extraction efficiency was achieved using a combination of solvent and sonication. Results demonstrate that continuous and non-destructive extraction of oils from live cultures at substantially reduced costs can be accomplished using bio-compatible solvents.

Besides the usable lipids already described, plant species such as algae are also known to produce important hydrophobic aromatic compounds. Some aromatic compounds such as naphthalene and toluene are important constituents in fuel products. Advantageously, the solvent extraction techniques described above may be used to extract many of these aromatic compounds as well as other useful oils previously described. These chemicals would not be extractable using current extraction techniques that rely on centrifugation and drying methods described above.

Although algal extraction is the focus of many of the exemplary embodiments, the growth and recycle extraction process may also be used with other important oleaginous organisms. For example, organisms such as yeast and fungi would also be amenable to this type of purification process.

In operation, cells may be grown in culturing systems and may be continuously pumped to a mixing chamber where they may be mixed with biocompatible solvents and sonicated under conditions previously determined to be optimal for maintaining cell viability and maximizing oil extraction. The cells/solvent mix may then pumped to a phase-separation chamber to allow the cells (lower phase) to partition from the solvent (upper phase). After the cells and solvent have partitioned, the cells may be recirculated back to the cell growth reservoir. The oil-containing solvent (upper phase) may be distilled (decane boiling temperature˜174° C.) and the lipid fraction will be quantified and characterized by GC-MS. The distilled solvent will be recirculated back to the algal extraction chamber and reused. A small fraction of the solvent is expected to partition into the aqueous phase. Since we will be gassing cells with air or CO₂-enriched air, we may be off-gassing some portion of the solvent. To determine the magnitude of this loss, the gas discharge may be collected and cooled using a refrigerated trap to condense and quantify any gassed-off solvent. Once the system is optimized, the energy consumed to operate the system using watt meters may be quantified. The solvent extraction of oils in exemplary embodiments disclosed herein may be highly efficient and low-cost.

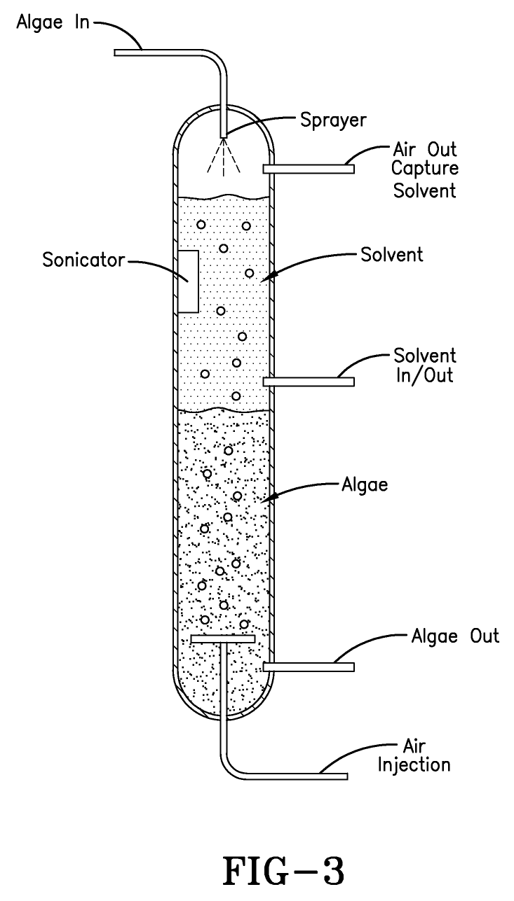

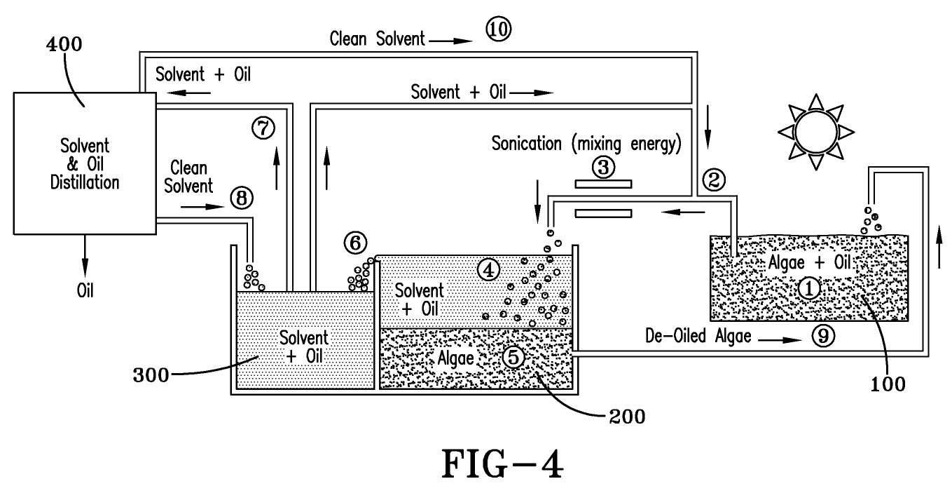

FIG. 3 shows a schematic model for a continuous flow solvent-based oil extraction system that complements the invention disclosure for solvent-based oil extraction. In one exemplary embodiment, the process may include: 1. Spraying the algae into the top of a long column to break up the droplet size for maximum mixing with the upper solvent phase. 2. The upper portion of the extractor may contain sufficient solvent (depth) to allow enough time during settling of the algae for complete oil extraction. 3. A sonicator element may be provided in the solvent phase to accelerate and improve solvent extraction of oils 4. Air may be injected into the algae phase intermittently to enhance mixing and to remove residual solvent from the algal phase. It may be advantageous to stop air injection during sonication to enhance the oil extraction 5. Plumbing may be provided for separate removal of the solvent and algal phases.

Columns like that shown in FIG. 3 may work individually or in parallel. When the solvent is oil saturated in one column then it may be shut down while the solvent is exchanged to recover the oil and sent to a distiller for removal of the solvent phase. In the meantime, algae may be pumped into the other columns.

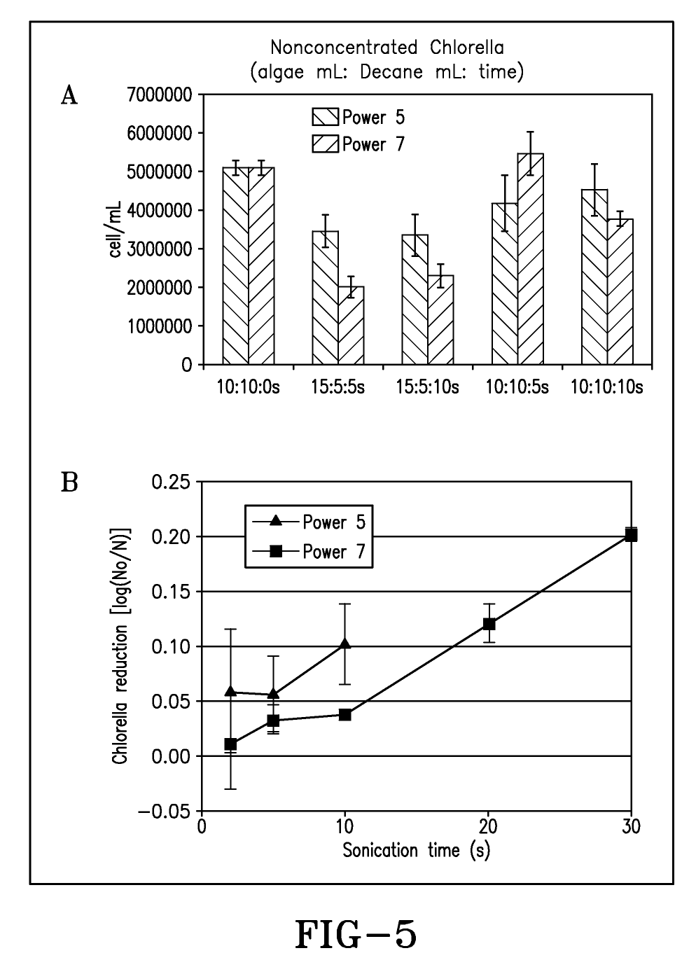

FIG. 4 illustrates another exemplary system and method for continuous flow, solvent-based oil extraction. As shown at point 1, an organism such as photosynthetic algae may be grown in an outdoor pond (100) where the culture may be exposed to solar radiation. As shown at point 2, a portion of the culture may be mixed with a solvent. Preferably, either mechanical mixing and/or sonication, may be used to improve mixing of the solvent and the organism (point 3). Sonication should endure for predetermined amount of time in order to maximize lipid extraction and minimize microorganism cell destruction. In the alternative, sonication may occur prior to exposing the culture to the solvent. The cells/solvent mix may then be directed to a phase-separation or partitioning chamber (200) to allow the cells (lower phase) to partition from the solvent (upper phase)(see point 4). As shown in points 4/5, the de-oiled cells and water may then sink to the bottom of the tank and the live cells may then be directed back into the pond to begin the process anew (point 9). The solvent and oil collected by phase separation may then float over a separation weir (point 6) into a solvent and oil chamber (300). As demonstrated in point 7, the solvent and oil may be directed into a distillation unit (400) (when the oil concentration is high enough for effective separation). At point 8, after the oil is removed, clean solvent may be pumped back in to the solvent tank for recirculation. Or in the alternative, the clean solvent may be recycled for mixing with the cell culture at point 2 (demonstrated by point 10).

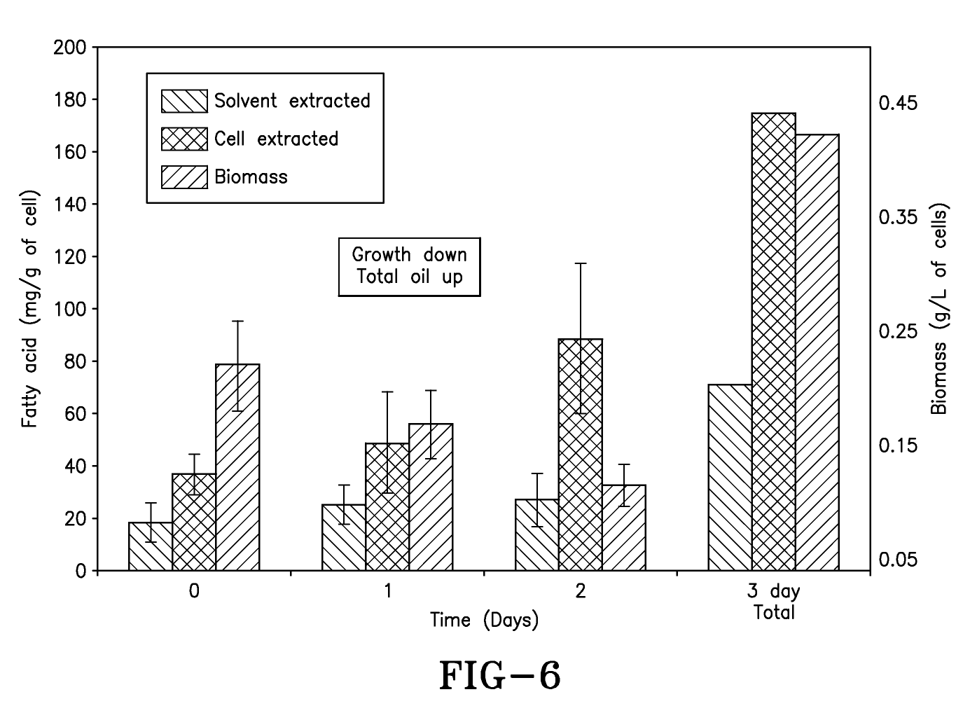

FIG. 5 displays the results of an experiment demonstrating the effect of sonication and decane extraction on viability of the green alga Chlorella protothecoides. Panel A shows the reduction of concentrated C. protothecoides viability after sonication using power 5 and 7 ultrasound up to 30 seconds and algae:decane volumetric ratio of 1:1. Reduction is calculated as log (No/N) where No is initial count of algae/mL and N is count after treatment. Panel B shows the impact of algae:decane ratio on cell death.

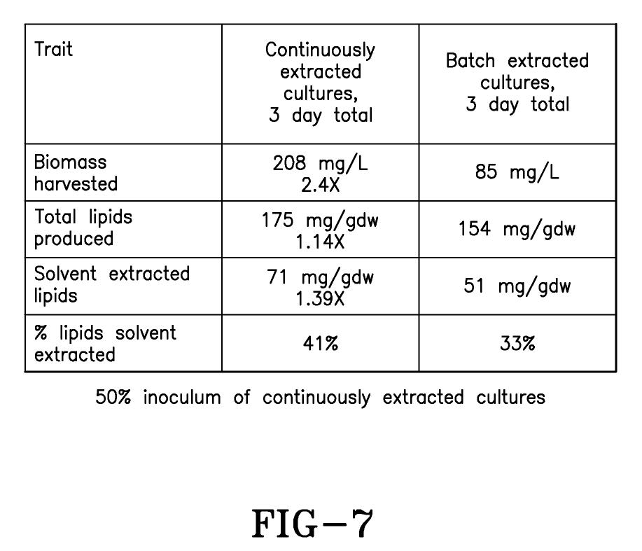

FIG. 6 graphically shows the results of an experiment demonstrating that repetitive solvent extractions may be performed to optimize the yield of energy rich molecules. In this experiment, repetitive solvent extractions with 50% inocula were performed. The data demonstrate that solvent extraction of live cells (C. protothecoides) removes triacylglycerols (represented as fatty acid equivalents) and that oil extractions can be made on a daily basis to recover more oil or neutral lipids. The total lipids (neutral and polar) in the cells are indicated by the middle bar of each group. The total neutral lipid (oil) extracted after two sequential extractions was equal to 20% of the total cellular biomass or 40% of the total cellular lipids (neutral and polar). There was a decrease in growth rate observed, however, after multiple solvent extractions.

In FIG. 7, data are shown that demonstrate repetitive solvent extraction yields more oil. The data represent a summary of total biomass and non-destructively extracted neutral lipids of daily versus batch (3^{rd} day only) extracted cultures. The results demonstrate a 2.4-fold greater increase in total biomass following sequential solvent extractions as well 41% increase in total oils extracted from daily extracted algae versus a 33% increase from batch treatment extracted algae of the same age. These results indicate that solvent extraction reduces growth inhibition as well as reduces the culture residence time to produce oil. These results indicate that the effective residence time in the pond to produce an equivalent volume of oil is nearly three times shorter for non-destructively extracted algae than for destructively extracted algae grown in batches.

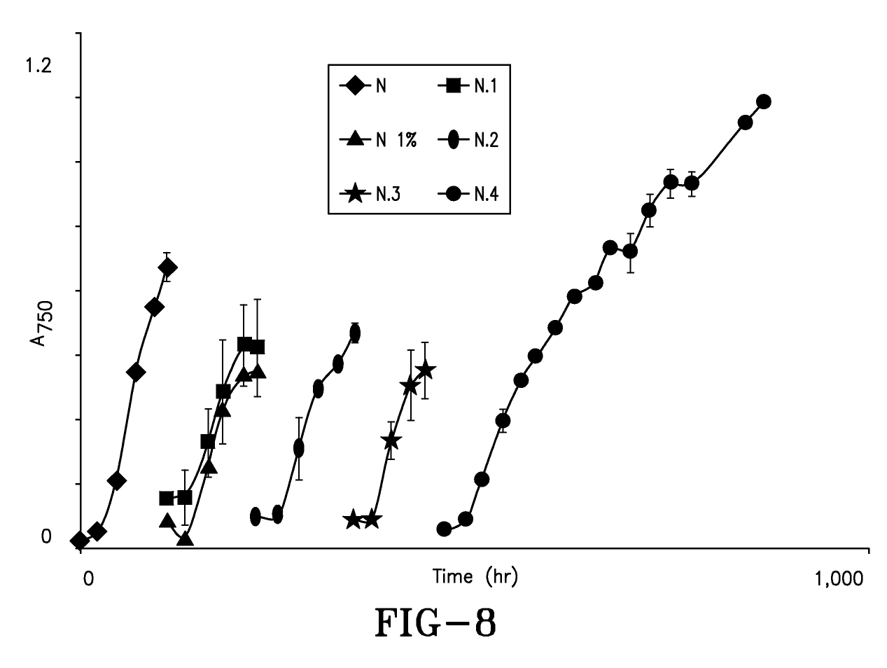

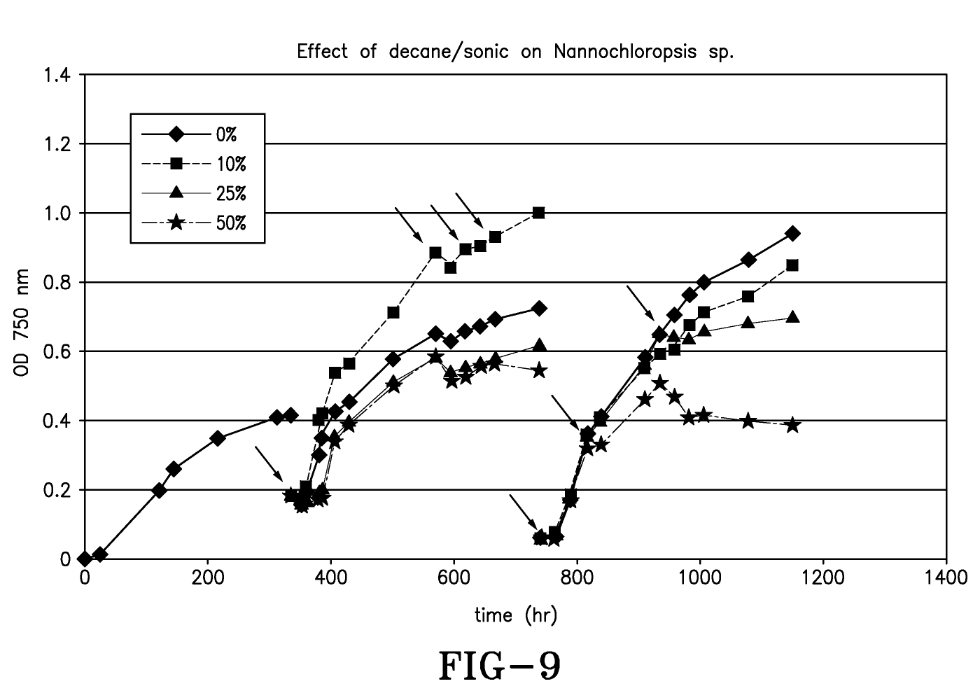

FIG. 8 contains data showing that growth of Nannochloropsis is not impaired after multiple cycles of non-destructive lipid extraction. These results demonstrate that Nannochloropsis sp. is more resistant to solvent extraction than C. protothecoides. The experiment shows grow out rates following solvent extraction as described in FIG. 2. Following four solvent extractions there was no impediment in growth rate. N=initial growth rate, no solvent extraction, start day 0; N1=growth rate after one solvent extraction, start day 1; N1%=growth rate of non solvent extracted cells, start day 2; N2=growth rate of cells solvent extracted a second time 24 hours later, day 2; N3=growth rate of cells solvent extracted a third time 24 hours later, start day 3; N4=growth rate of cells solvent extracted a fourth time 24 hours later, start day 4.

The above embodiments are exemplary. A wide array of devices and procedures may be used to achieve solvent-based oil extraction. For example, the algae culture and the solvent may be caused to flow as counter current flows. Alternatively, bubble chambers may be useful for mixing. Other designs utilizing a screw-like chamber to force the mixing of the algae and the solvent may also be used for efficient mixing.

### EXAMPLES

In order to facilitate a more complete understanding of the invention, a number of Examples are provided below. However, the scope of the invention should not be limited to the specific embodiments disclosed in these Examples, which are for purposes of illustration only.

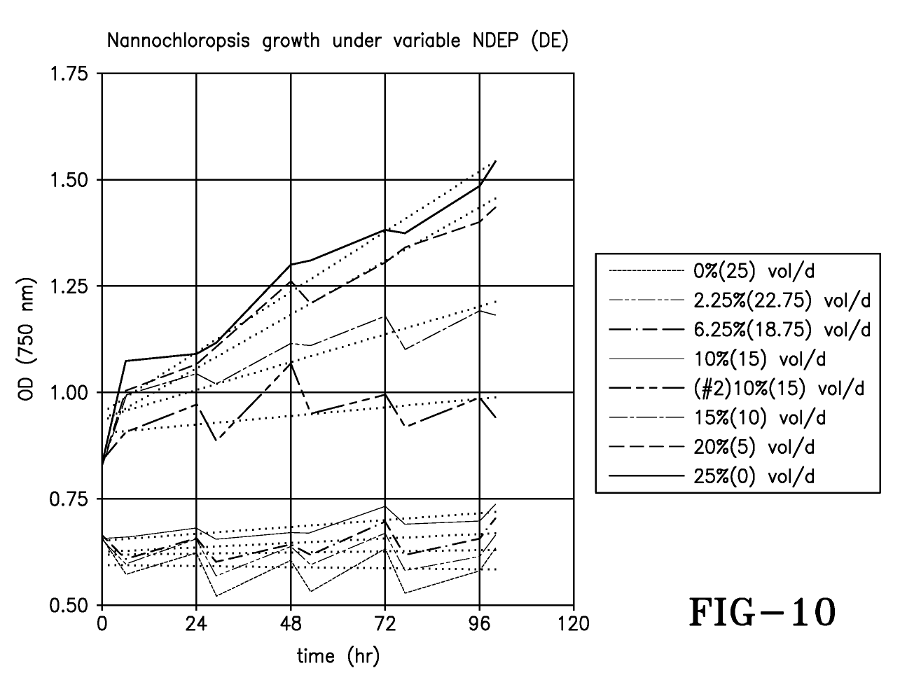

### Example 1 (FIG. 9)

### Effect of Decane and Ultrasonic Treatment on Nannochloropsis

Variable fractions (0, 10%, 25%, and 50%) of identical 100 mL Nannochloropsis sp. cultures (n=2) were initially (arrows) mixed (15 min) with decane and exposed to an ultrasonic field (2 sec; 40 kHz water bath), decanted of solvent, then grown (F/2, 23° C., 24:0 of 100 μmol, 100 rpm, 33 ppt, 100 mL in 500 mL flasks). Further, variable timed treatments (arrows) at log-phase and stationary phases were also completed. This figure shows some levels of exposure can positively affect growth rate and resultant algal biomass, as compared to no treatment. Further, stationary phase cultures are generally more tolerant to solvent-sonic treatment than log-phase cultures, however this effect may be more related to the higher cell concentrations than to the specific physiological life-stage.

### Example 2 (FIG. 10)

### Decane/Sonic Extraction Effect on Extended Growth of Nannochloropsis

Under simulated outdoor growing conditions (30 ppt, 26-37° C., 14:10 of 1000 umol, F/2) 12 liter aquaria of Nannochloropsis sp., equipped with mixers and pH controlled (˜7.2) CO₂ gas input, had 25% of culture volume removed daily which was variably (0-25% of total fraction) extracted of lipids with decane (15 min) and sonic (2 sec) energy, decanted of solvent, then returned to culture, while the remaining untreated fraction (0-25%) was removed, dried and extracted with hexane. The figure shows daily exposure to treatment is tolerated, that cultures with higher initial cell concentrations perform better (positive growth), and that increasing levels of decane/ultrasonic exposure up to 25% per day of the culture volume augment growth rates and the resultant culture biomass.

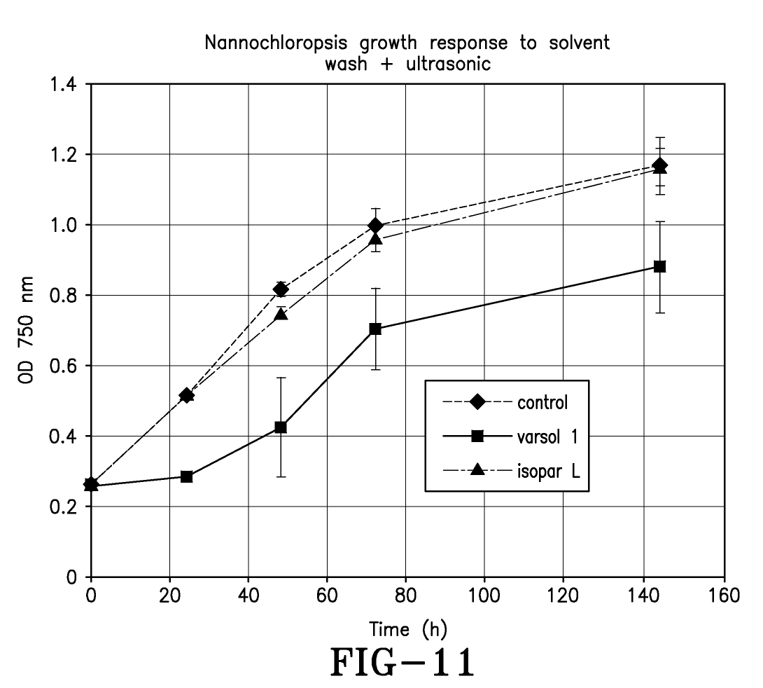

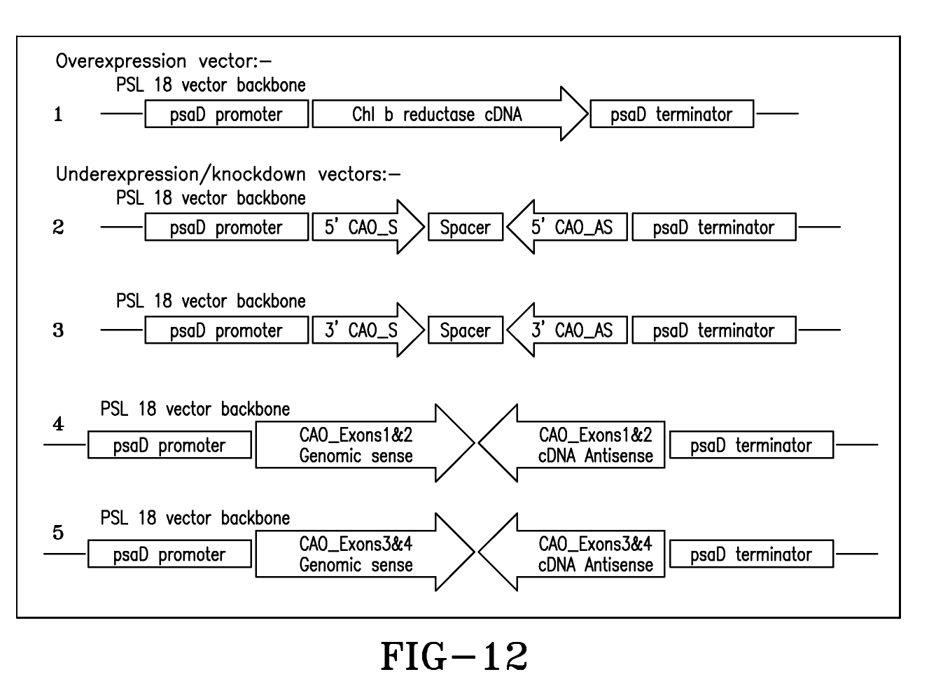

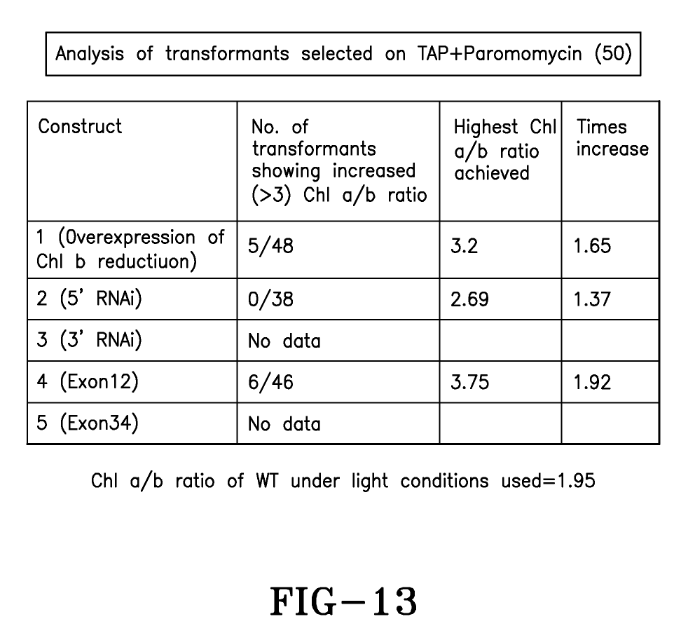

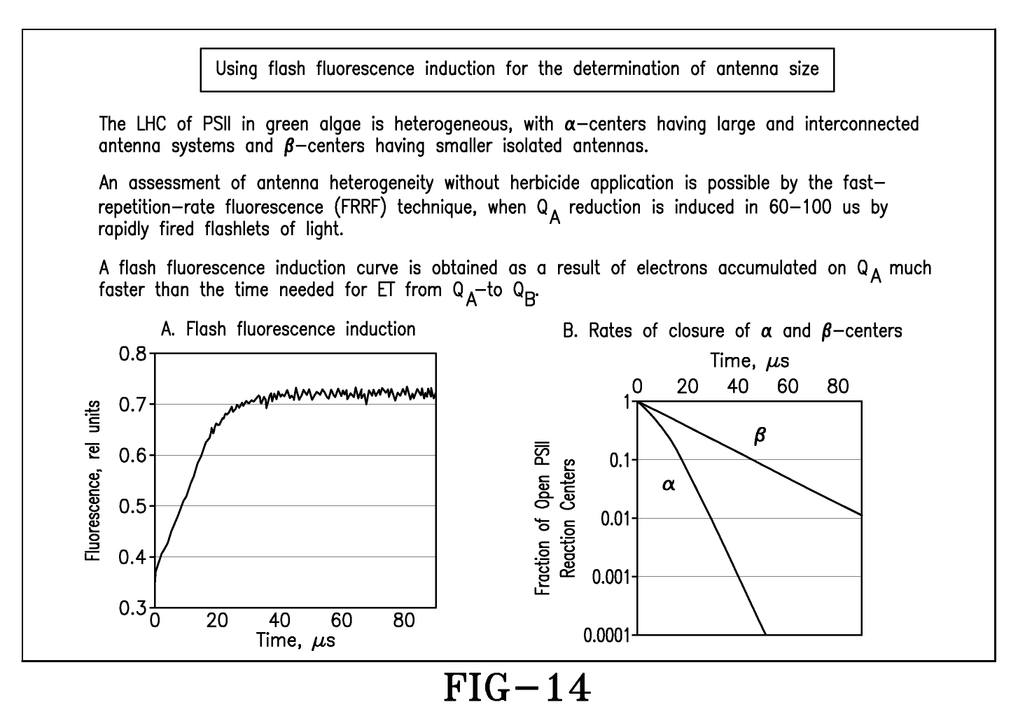

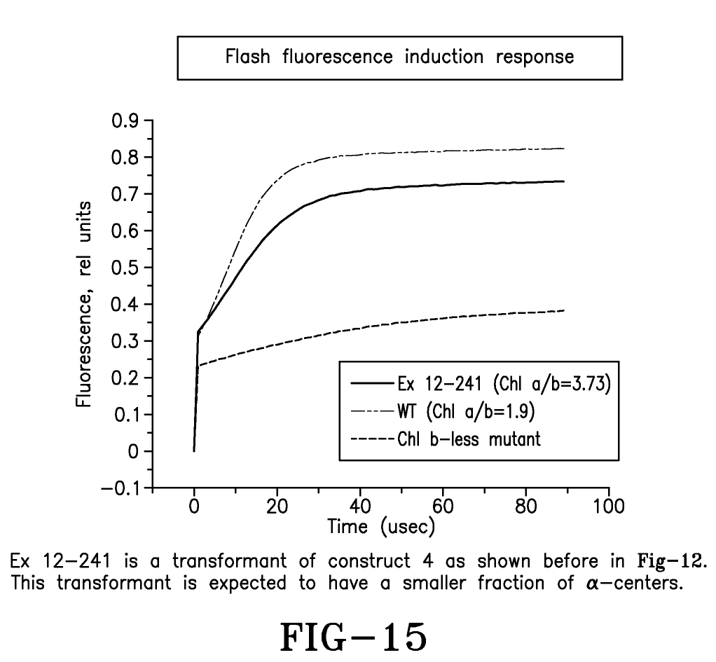

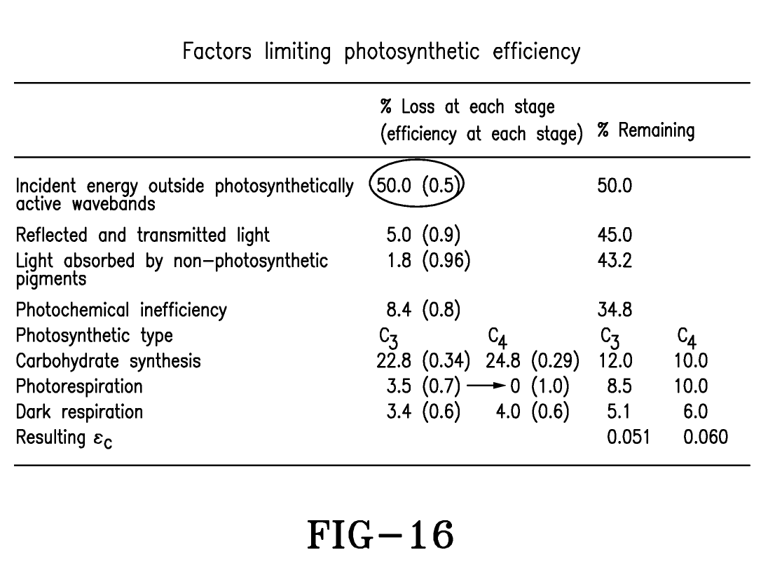

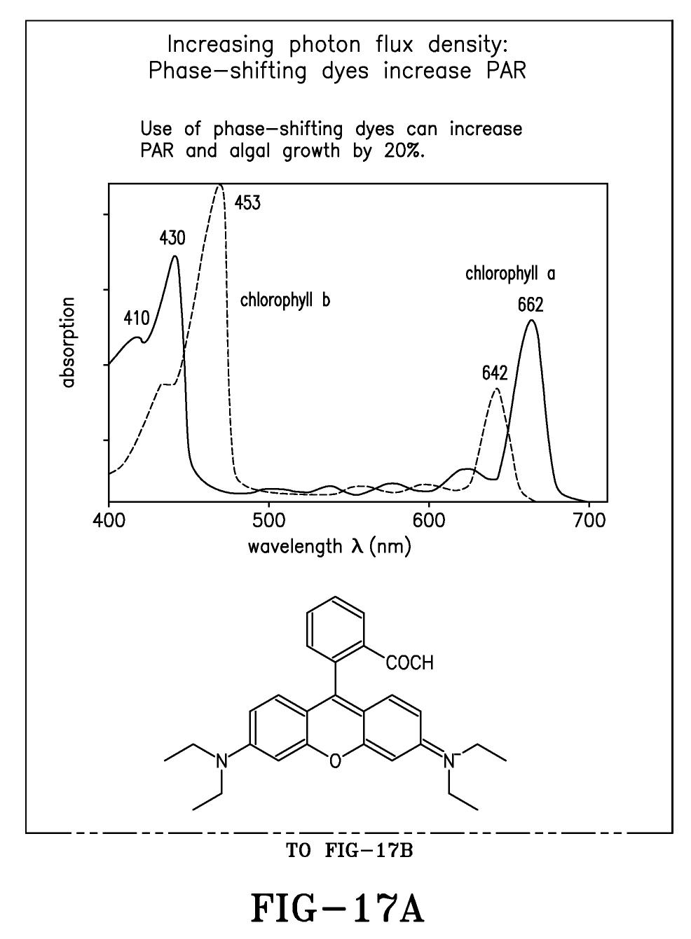

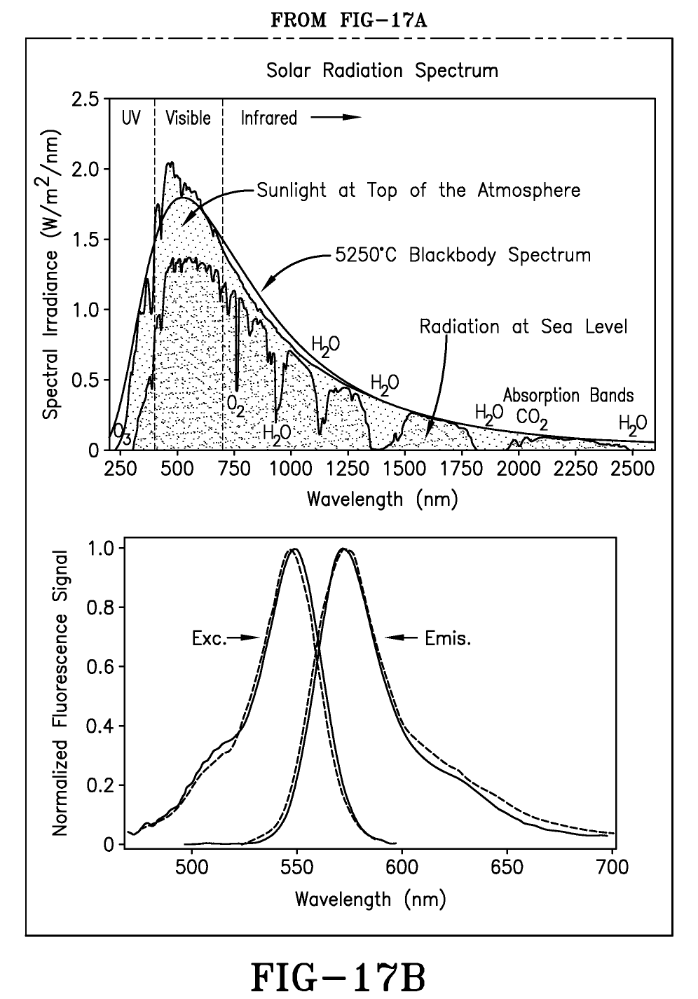

### Example 3 (FIG. 11)

### Extraction with Economical Solvents

Identical cultures (n=2) of Nannochloropsis sp. (100 mL, 26 C, 80 umol, F/2) were treated to an initial exposure (15 min) of an economical alternative extraction solvent (Varsol 1 (cyclic paraffin), Isopar L (parraffin) an EXXON product obtained through Univar) and ultrasonic energy (2 sec, 40 kHz), then grown for 144 hours. The figure shows that algae exposed to isopar L and varsol 1 possessed growth rates nearly identical to untreated control, meriting their applicability in non-destructive extraction processes.

### Example 4

### Nondestructive Solvent Extraction Procedure with Yeast Materials

Red Star dry active baker's yeast (Saccharomyces cerevisiae)
Yeast Extract Proteose Dextrose medium (ATCC #1245)
Isopar L (EXXON through Univar)

Procedure and Results:

One gram of dry yeast was added to 200 mL of Yeast Extract Proteose Dextrose medium (YEPD) and incubated at room temperature with 200 RPM shaking for overnight. Ten mL of this culture was added to 150 mL of YEPD and grown as above. Then 20 mL of this overnight yeast sub-culture was combined with 20 mL of Isopar L and vortexed. Then the well mixed sample in a 250 mL Erlenmeyer flask was briefly sonicated and transferred to a 50 mL tube to facilitate solvent separation. Before extraction, one mL of the overnight culture was added to 8 mL of YEPD in a 15 mm×100 mm test tube and incubated overnight. One mL of the after solvent exposure was added to 8 mL of YEPD in a 15 mm×100 mm test tube and incubated overnight. The optical densities of pre-exposure and post-exposure cultures at 750 nM (A₇₅₀) were measured after the overnight incubation. A₇₅₀ of pre-solvent exposure cultures: 1.66; 1.67. OD of post-solvent exposure cultures: 1.83; 1.88. Similar A₇₅₀s of the pre- and post-solvent exposure indicates solvent exposure similar to the non-destructive extraction procedure does not diminish the growth capacity of the yeast.

### Example 5

### Species Screen of Various Algae for Solvent Stability

Similar to Example 4, Table 1 contains data showing that solvent extraction had similar effects in other strains.

TABLE 1

| | | | | | | | Percent Dead | | | |
|---|---|---|---|---|---|---|---|---|---|---|
| | | | | OD | | | Dead cells/field (pre | | | Dead/ |
| UTEX # | | Strain | Salinity | 0 h | 30 h | 64 h | solv/son) | | | post |
| 1230 | A | Chlorella | IO/3 | 0.654 | 0.739 | 0.832 | 0/100, | 0% | 0/396, | 0% |
| | | sorokiniana | | | | | 0/100 | | 0/276 | |
| 1602 | B | Chlorella | None | 0.513 | 0.482 | 0.586 | 1/202, | 0% | 0/215, | 0% |
| | | sorokiniana | | | | | 0/121 | | 0/163 | |
| 2164 | C | Nannochloropsis | None | 0.143 | 0.708 | 0.371 | 0/69, | 1% | 0/71, | 0% |
| | | oculata | | | | | 1/67 | | 0/85 | |
| 2229 | D | Chlorella | IO/3 | 0.147 | 0.076 | 0.133 | 9/72, | 14% | 0/8, | 0% |
| | | Kessleri | | | | | 8/51 | | 0/8 | |
| 2341 | E | Chlorella | IO/3 | 0.517 | 0.556 | 0.749 | 1/244, | 1% | 0/282, | 0% |
| | | minitissima | | | | | 4/505 | | 0/239 | |
| 2805 | F | Chlorella | None | 0.484 | 0.656 | 0.709 | 1/110, | 0% | 0/76, | 0% |
| | | sorokiniana | | | | | 0/154 | | 0/99 | |
| 25 | G | Chlorella | IO/3 | 0.913 | 0.945 | 1.171 | 21/193, | 15% | 30/465, | 7% |
| | | protothecoides | | | | | 28/136 | | 15/174 | |
| — | H | Nannochloropsis | IO/3 | 0.696 | 0.797 | 0.947 | 5/272, | 3% | 0/232, | 0% |
| | | sp. | | | | | 12/379 | | 0/193 | |
| 1602 | I | Chlorella | IO/3 | 0.237 | 0.323 | 0.416 | 0/118, | 0% | 0/31, | 0% |
| | | sorokiniana | | | | | 0/84 | | 0/74 | |
| 2164 | J | Nannochloropsis | IO/3 | 0.420 | 0.604 | 0.856 | 2/420, | 0% | 0/283, | 0% |
| | | oculata | | | | | 1/279 | | 0/269 | |

### Example 6

### Post-Treatment to Remove Emulsion

Although capable of accelerating the extraction of lipids from cells, solvents in aqueous solutions often form very stable emulsions when exposed to ultrasonic energy or vigorous mixing. This clouding (emulsion) of the aqueous solution is created by the nebulized solvent which does not easily coalesce, even after lengthy settling periods. Those skilled in the art utilize methods to accelerate the separation of solvent from the aqueous fraction. These include use of microfiltration (eg., borosilicate microfiber), ultrasound standing waves, coalescing media, hydrocyclones, addition of flocculating agents (e.g., aluminum) or gas floatation. These methods vary in speed and efficiency but will selectively remove trace solvents from the aqueous solution, allowing its recapture, and prevent potential system losses. For example, an emulsion of solvent in water (0.03%), quantified by its reduction of light transmission through a 1 cm light path at 350 nm, was clarified from 75% to 100% light transmission by microfiltration of the emulsion, effectively coalescing the solvent.

### Example 7

### Distillation of the Nannochloropsis Oil from the Solvent

An extraction mixture of solvent (Isopar L) and extracted solute (Nannochloropsis algal oil) was removed from the effluent solvent tank of the non-destructive extraction process pilot system (NDEP). The volume of the Isopar L and algal oil mixture was then measured. Next, this mixture was placed into a round bottomed flask and attached to a Buchi 210/215 rotary evaporator (Rotovap). Cold tap water was run through the condenser and an oil bath for the distillation flask was set to 140° C. Once the oil bath reached 140° C., a vacuum of 85 mbar was drawn on the whole system. The intention of the high temperature and low pressure within the Rotovap is to exploit the vapor pressure discrepancy between Isopar L and the algal oil. When distillation began, gaseous Isopar L traveled through the instrument to the condenser then returned to a liquid state that was collected in the receiving flask. While the initial distillation parameters (140° C. and 85 mbar) were sufficient to start evaporation of Isopar L from the distillation flask, these conditions were insufficient for complete distillation of the Isopar L from the algal oil. This could be due to the nature of Isopar L as a mixed solvent versus a single component. When distillation began to slow, as observed by the lack of condensate, the vacuum in the Rotovap was increased by 5 mbar increments until distillation began again. This procedure of increasing the vacuum was repeated every time it was noticed distillation had either stopped or slowed until a final vacuum of 35 mbar was reached. At the end of the experiment, the volume of Isopar L recovered in the receiving flask was measured, as well as the volume of algal oil left within the distillation flask.

### Example 8

### Recovery of Lipids from Extraction Media

The lipids contained in certain strains of algae have value as transportation fuels and other energy applications. These lipids must be grown, harvested, and then purified/concentrated to have economic value. Prior to the purification and extraction process it may be necessary to condition the algae for improved extraction efficiency. This process is highly variable and would be similar to oil seed conditioning which is described in detail in US patent application US2008/0269513. Key in this cycle are the purification and concentration steps. Several different methods are suitable for removal of the extracted lipids from the solvents used in this invention.

Adsorbents that use surface phenomena to bind the extracted lipid and then are treated to release the lipid when desired are used to efficiently remove the lipid from the solvent. The absorbents can be activated carbon, alumina, silica gels, molecular sieves and the like. The lipid is removed by a pressure and or temperature cycle and the absorbent reused for further extractions.

Lipids may also be extracted using a fluids/mixture treatment with temperature and pressure. This technique relies on the relative differences of the physical properties of the extracting solvent and the lipids being purified. Commercial examples of this include crystallization, solute exclusion and ternary extraction. The fact that lipids and the candidate solvents (e.g., decane, dodecane, ISOPAR, Varsol) have wide miscibility ranges allows use of partially saturated extraction fluids make this a viable route for purification.

Reverse osmosis and semi-permeable membranes are often used for separation of chemicals based on solubility or actual molecular size. These allow the solvent or the lipid to pass through them preferentially effecting efficient separation of the solvent and solute. This technique is similar for both liquids and gases and is described in some detail in US Patent Application 20080141714 for the purification of natural gas. The system envisioned here for separation of biocompatible solvent and extracted lipid is similar in function and equipment requirements.

Vapor compression distillation can be used for any two component liquid mixture where separation is desired. The system achieves high efficiency (low cost) through the use of vapor compression in conjunction with multiple heat exchangers. This method is described in detail in U.S. Pat. No. 4,539,076.

Vacuum distillation can be used in combination with vapor compression distillation in cycle where it is desired to accomplish separations at reduced temperatures thereby reducing the thermal degradation of one or more of the components being separated. This technique is well established and described extensively in the literature.

Any of the above purification methods may be combined to affect a more complete separation of solvent and solute (algal oil) in a stepwise fashion.

Enhancing Lipid Yield Through Increasing Photosynthetic Efficiency:

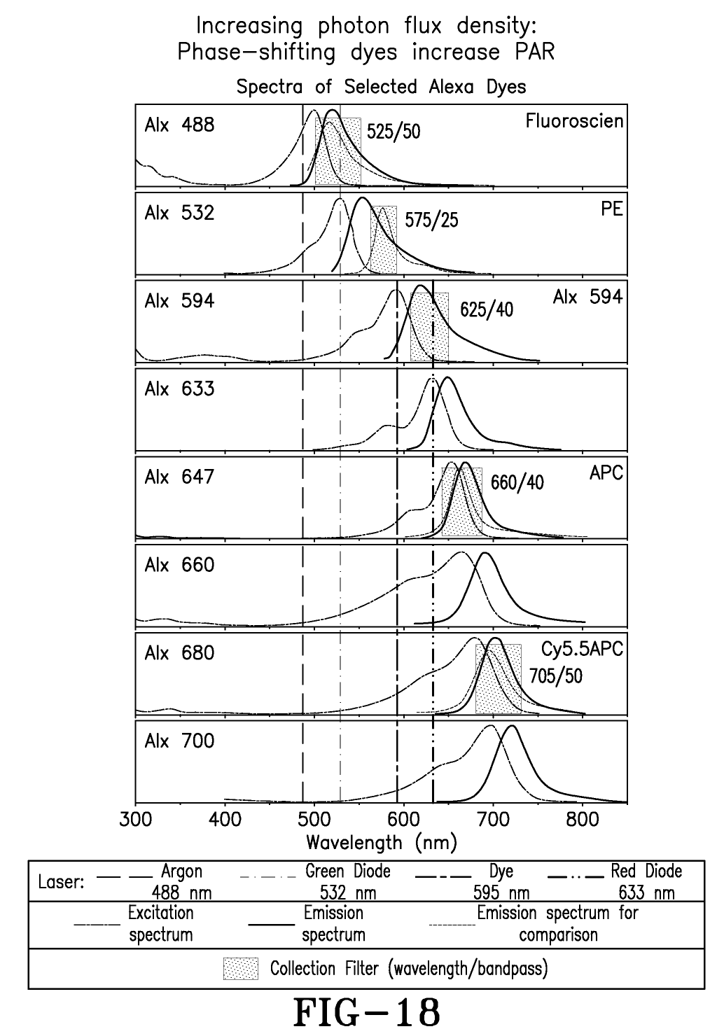

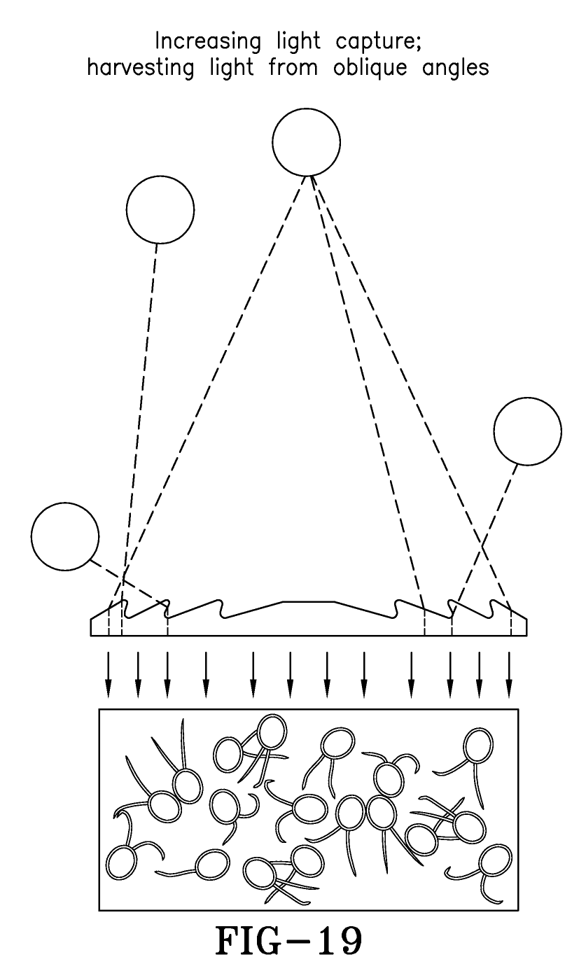

The major factor limiting photosynthetic efficiency and thus crop or biomass productivity is the inability of chlorophyll to absorb over 50% of the available solar energy present at the earth's surface (FIG. 18). A major window of visible light ranging between 400 and 600 nm is not absorbed by chlorophyll (FIG. 19). To overcome this limitation, some photosynthetic organisms (cyanobacteria and red algae) synthesize additional light-harvesting accessory pigments including carotenoids and phycobiliproteins that harvest light between 400 and 600 nm. These pigments transfer absorbed energy to chlorophyll by resonance energy transfer mechanisms. Plants and most eukaryotic algae, with the exception of the red algae, lack these accessory pigments and do not efficiently absorb light between 400-600 nm.

Exemplary embodiments address this limitation in light harvesting by absorbing the normally unused light (e.g., light between 400-600 nm) and emitting this energy at more usable wavelengths (e.g., such as between 650-680 nm). Preferably light emissions will be largely in the red region of the chlorophyll absorption spectrum. While chlorophyll absorbs light both in the blue and red portion of the spectrum it is the lowest excited state corresponding to excitation in the red that drives photochemistry in photosynthesis. Thus, small losses of energy due to vibrational and non-radiative processes associated with energy transfer between dyes and their fluorescence emissions do not dramatically affect the efficiency of the system.

A series of dyes (as exemplified by the example dyes shown in FIG. 18) with overlapping excitation and fluorescence emission spectra may be embedded in films at concentrations high enough to optimize energy transfer between the most blue light (e.g. Alexa 488) and red light (e.g., Alexa 660) absorbing pigments. Light may be emitted by the lowest energy fluorochrome (e.g., Alexa 660) and the emission of this light will be matched to the red absorption spectrum of chlorophyll (620-690 nm). The increase in the number of photons harvestable by photosynthetic organisms, particularly at light intensities that do not saturate the photosynthetic machinery, will increase photosynthesis and biomass yields. These films can be placed over plants or in bioreactors to enhance photosynthetic light harvesting efficiency. In addition, dyes may be incorporated into Fresnel lenses that focus ambient light on the culture. Organisms that have been engineered (e.g., by elimination of the chlorophyll a/b light harvesting complex) to have higher light saturation optima for photosynthesis are likely to show the greatest improvement in photosynthetic efficiencies using this technology.

In some exemplary embodiments, the wavelength shifting dyes may be incorporated into particles that may be suspended in the growth media. This has the advantage of re-radiating the wavelength-shifted light in all directions to be captured by the algae. In contrast a bioreactor cover, with wavelength shifting dyes, may lose 50% of the wavelength shifted light due to re-radiation back into the atmosphere. The particles could be made ferromagnetic so that they can be extracted easily from the culture prior to solvent extraction.

### EXAMPLES

In order to facilitate a more complete understanding of the invention, a number of Examples are provided below. However, the scope of the invention should not be limited to the specific embodiments disclosed in these Examples, which are for purposes of illustration only.

### Example 9

### Effect of UV Absorbing Dyes Embedded in Polycarbonate to Modulate the Light Quality for Algal Growth

Polycarbonate with an embedded dye (high blue #368D, Bayer Material Science LLC) can be used to filter natural sunlight onto flasks containing algae growing in a photoautotrophic medium. This dye shifts ultraviolet light (300-400 nm), which chlorophyll does not absorb, into the blue range that can be utilized more efficiently by the chlorophyll in algae for photosynthesis.

### Example 10

### Effect of UV Absorbing Dyes in Solution to Modulate the Light Quality for Algal Growth

Alternatively, the wavelength-shifting filter is not dye-embedded polycarbonate, but instead a fluorescent dye (such as Alexa Fluor 647, Molecular Probes) dissolved in a buffer and contained in a reservoir made of plexiglass. In this case, the dye shifts yellow and orange light (and to a lesser extent, green light) to a range of red light absorbed most effectively by chlorophyll. The edges of the reservoir are sealed such that the only light that reaches the culture passes through the dye solution.

### Example 11

### Growth of Algae in the Presence of Magnetic Particles Coated with Light Shifting Dyes

In another embodiment, the dye may be incorporated into (or onto the surface of) a magnetic particle. For example, the succinimidyl ester form of Alexa Fluor 647 may be conjugated to small paramagnetic beads via a carboxamide linkage. The beads are then added to the culture flask with the algae. Cultures can be grown in omnidirectional light (i.e., not in a light box) and mixed by shaking or stirring. The beads may be drawn away from the algal culture magnetically before withdrawing samples.

The above-described dyes enable the culture to grow faster proportional to the ability of the wavelength-shifting dye to absorb wavelengths of light not used efficiently for photosynthesis and emit blue or red wavelengths absorbed most efficiently by chlorophyll. The cultures should be mixed or aerated vigorously enough to prevent CO₂ limitation. In some examples, light intensity should be kept close to 200 mmol m⁻² sec⁻¹ to maximize growth without saturating the photosynthetic apparatus and overwhelming the effect of the wavelength-shifting dye.

Light availability will be impacted directly by the position and angle of the sun. Photosynthetic organisms may not be able to capture as much energy from light entering at oblique angles. However, exemplary embodiments overcome low light fluence using engineering solutions which may increase the light fluence levels in photo-bioreactors. FIG. 19 illustrates one method that may be used to enhance light fluence. In FIG. 19, a Fresnel lens is utilized to enhance the collection of light when the light source is received at oblique angles. Additional devices, such as collecting mirrors, may also be used to enhance light fluence levels in algae lacking the LHC complex.

### Example 12

### Method for Attaching Fluorescent Dyes to Magnetic Beads for Light Frequency Shifting Experiments

The attachment of fluorescent dyes that absorb light in the 400 to 600 nm range to plastic beads or plastic coated paramagnetic beads is to improve the photosynthetic efficiency of algal cells by the beads capturing poorly used light wavelengths and remitting fluorescence in the 650 to 690 nm region optimal for algal photosynthesis. These beads are then retrieved after use so that they can be reused or recycled. If the beads are rather large they can be filtered out, however filtering is not an efficient process, requires periodic replacement of clogged filters, and would have a higher shading effect than small beads. By using paramagnetic beads, the beads can be retrieved from a liquid state with high efficiency with a permanently magnetized material or electromagnet. Similar sorting processes are common in several molecular biology techniques, including nucleic acid capture, in vitro display, immunoprecipitation, and his-tagged protein purification.

Several sources of paramagnetic beads with modified surface groups are readily available. Dynabeads (Invitrogen) are a good example because they are uniform in size and shape, offer a variety of surface modifications, three size ranges (1, 2.8 and 4.5 um), and are offered in bulk for industrial applications. They offer hydrophobic or hydrophilic surface characteristics with epoxy-, amine-, tosyl-, and carboxylic acid-surface groups. Each surface modification has its own ligand specificity and coupling buffer. See the table below for relevant surface modifications and reactive ligands. Additionally they provide beads that have terminal amine groups (Dynabeads M-270 Amine) that can be used with SH-reactive agents such as NHS-esters.

TABLE 2

| Surface | | |
|---|---|---|
| modifi- | | |
| cation | Ligand specificity | Coupling buffer |
| Amine- | Aldehyde or ketone groups | Activated with NHS-ester then |
| | | coupled in 0.1 M Phosphate |
| | | buffer with 0.15 M NaCl pH 7.4 |
| Epoxy- | Amine, hydroxyl, and thiol | PBS with 1-3 M ammonium |
| | groups | sulfate |
| Carboxyl- | Primary Amine | Activated with 0.2 M EDCI then |
| | | coupled in 50 mM MES pH 5.0 |
| | | buffer |
| Tosyl- | Amine, and sulfhydril | 0.1 M sodium phosphate buffer |
| | groups | pH 7.4 with 3M ammonium |
| | | sulfate |

In Table 2; Invitrogen's surface modified paramagnetic beads (Dynabeads®) and related chemistries. Abbreviations: PBS, Phosphate buffered Saline; EDCI, 1-ethyl-3-(3-diethylaminopropyl)carbodiimide hydrochloride; MES, 2-(N-morpholino)ethanesulfonic acid; NHS, (N-hydroxy-succinimidyl)-ester;

To bind ligands (modified fluorescent dyes) to beads, the beads are washed in their respective storage buffer. This step is followed by activation (if necessary) in coupling buffer containing their respective activating reagent for up to 30 minutes. The beads are then washed several times in coupling buffer, then mixed with the dyes suspended to the appropriate concentration and volume in their respective coupling buffer. The dye/bead mixture is then incubated for several hours to overnight at room temperature with frequent inversion. The beads are then magnetically separated from the coupling buffer and washed several times with fresh coupling buffer without the dye. This is subsequently washed one more time in an appropriate storage buffer depending on the dye's requirements.

Additional coating of the beads can be achieved by simple washing and incubating in the desired solutions. For instance it may be necessary to coat the fluorescently labeled beads with a hydrophobic layer to prevent oxidation of the dye. This can be achieved by incubating the beads in a hydrophobic solution such as Rain-Coat® or Dow's Hypod™ polyolefin. These are fluidized emulsions which allow materials to be sprayed or dipped into the suspension for even coating. After the beads are coated with the hydrophobic solution they can be washed again and stored dry or in an appropriate buffer at room temperature in the dark for long periods of time.

One could use the Alexa 488 fluoresecent dye that is preactivated with a succinimidyl ester (Molecular Probes cat. #A20000). This dye (3 ug) is mixed with 10⁷ beads to a final concentration of 1−2×10⁹ beads per mL. The Dynabeads M-270 amine need to be prewashed as directed by the manufacturer. Briefly the are resuspended by vortexing or rapid pipetting then transferred to the reaction vessel. The beads are collected with a magnet to the side of the vessel and the liquid removed. The reaction buffer (0.1 M sodium phosphate buffer with 0.15 M NaCl, pH 7.4) is added and the beads vortexed or rapidly pipetted again. The buffer is separated from the beads using the magnet and buffer decanted. The washed beads are brought to the correct volume such that, when mixed with the Alexa 488 NHS ester they will be at 1−2×10⁹ beads per mL. Incubate for 30 min at room temperature with slow tilting motion of the vessel to maintain mixing. After this incubation place on the magnet to separate the unreacted dye from the labeled beads and discard buffer solution. Wash the coated beads in 0.05M Tris pH 7 for at least 15 minutes to quench unreacted NHS at room temperature, again with slow tilting mixing motion. Wash in phosphate buffered saline (PBS) or equivalent buffer four times. Resuspend in buffer with a little surfactant, such as NP-40 to prevent clumping. These can be stored at low temperature until use. Long term storage should be with preservative addition such as sodium azide at 0.02%.

Another dye that is suitable for this is the Alexa 660 dye (Molecular probes cat. A20007) which absorbs in another region not useful for photosynthesis but emits in an are useful for chlorophyll absorption. This comes also as an NHS ester and can be reacted as for Alexa 488 described above.

### Example 13

### Method for Producing Non-Magnetic Beads

The equipment needed for the blending of clear polymeric material consists of a single or double screw multi-jacketed extruder with injection ports for the introduction of gaseous additives. After extrusion thru a single or multi-port die the expanded strands are feed into a water bath where they are cooled. The strand size is controlled by a variable speed belt which functions as a strand puller and pelletizer feeder. The hardened pellets would have the proper ratio of the two (or more) organic dyes embedded in the polymer and the gas would be controlled to achieve the needed buoyancy desired.

Feed hoppers are needed at the front end and metering screws would feed the dyes into a metered polymer stream where they would be pre-blended and fed into the extruder. The gas is fed into the extruder towards the end of the extruder where the polymer and dyes are molten and homogeneous.

This process equipment is similar to an Alcoa subsidiary called Alcan located in Glaskow Ky. They process virgin polystyrene with carbon black, reground off-spec product and other additives in a twin screw extruder and inject isopentane. The expanded foam board is feed continuously to be air cooled and laminated. The final product is a lightweight white board for erasable marker presentations.

Publications

The following references and others cited herein but not listed here, to the extent that they provide exemplary procedural and other details supplementary to those set forth herein, are specifically incorporated herein by reference in there entirety.
Ahmed F I and Russell C (1975). Synergism between ultrasonic waves and hydrogen peroxide in the killing of micro-organisms. J Appl Bacteriol 39, 31-40.
Becker E W (1994) Microalgae biotechnology and microbiology. Cambridge Univ. Press. New York, N.Y.
Brown L M (1996) Uptake of carbon dioxide from flue gas by microalgae. Energy Conversion and Management 37: 1363-1367. 36
Doucha, J., and Livansky, K. (2006). Productivity, CO2/O2 exchange and hydraulics in outdoor open high density microalgal (Chlorella sp.) photobioreactors operated in a middle and southern European climate. Journal of Applied Phycology 18, 811-826.
Hejasi, M. A. and Wijffels, R. H. (2004). Milking of microalgae. Trends in Biotechnology, 22, 189-194.
Hejasi, M. A., de Lamarliere, C., Rocha, J. M. S., Vermue, M., Tramper, J., and Wijffels, R. H. (2002). Biotechnology and Bioengineering 79, 29-36.
Hejasi, M. A., Holwerda, E., and Wijffels, R. H. (2004). Milking microalga Dunaliella salina for beta-carotene production in two-phase bioreactors. Biotechnology and Bioengineering 85, 475-481.
Kadam, K. L. (1997). Power plant flue gas as a source of CO2 for microalgae cultivation: Economic impact of different process options. Energy Conversion and Management 38, S505-S510.
Melecchi M I S, Peres V F, Dariva C, Zini C A, Abad F C, Martinez M M and Caramao E B (2006). Optimization of the sonication extraction method of Hibiscus Tiliaceus L. flowers. Ultrason Sonochem 13, 242-250.
Miao, X., and Wu, Q. (2006). Biodiesel production from heterotrophic microalgal oil. Bioresource Technology 97, 841-846.
Richmond A (2004) Handbook of microalgal culture biotechnology and applied phycology. Blackwell Publishing, Ames, la.
Sheehan, J., Dunahay, T., Benemann, J., and Roessler, P. (1998). A look back at the U.S. Department of Energy's aquatic species program-biodiesel from algae. National Renewable Energy Laboratory, Golden, Colo.
Tiehm A (2001) Combination of Ultrasonic and Biological Pollutant Degradation. In Advances in Sonochemistry: Ultrasound in Environmental Protection Vol. 6, Mason T J and Tiehm A, Eds., JAI Press: Stamford, Conn., 25-58.
Toma M, Vinatoru M, Paniwnyk L and Mason T J (2001) Investigation of the effects of ultrasound on vegetal tissues during solvent extraction. Ultrason Sonochem 8, 137-142.
Wase D and Patel Y R (1985). Effect of cell volume on disintegration by ultrasonics. J Chem Tech Biotechnol 35B, 165-173.

### Patents Referred to:

US patent application 2008/0269513 Integrated process for the preparation of fatty acid methyl ester (biodiesel). Swaroop Sarangan and Vidhya Rangaswamy; assignee Reliance Life Sciences PVT LTD.
US Patent Application 2008/0141714 Molecular sieve and membrane system to purify natural gas. Gordon T. Cartwright and Keith R. Clark

1. A method for oil extraction from an oleaginous organism, comprising:
mixing at least a portion of a culture containing an oleaginous organism with a solvent that extracts oil from the oleaginous organism to obtain a solvent-organism mixture;
directing the solvent-organism mixture into a partitioning chamber to obtain an extracted aqueous fraction containing a viable extracted organism and a solvent-oil fraction; and
recycling the viable extracted organism into a culturing system.

2. The method of claim 1, wherein the method further comprises the step of:
distilling the solvent-oil fraction to obtain a usable oil.

3. The method of claim 1, wherein the method further comprises the steps of:
distilling the solvent-oil fraction to obtain a usable oil and recovered solvent; and
recycling at least a portion of the recovered solvent for use in the mixing step.

4. The method of claim 1, wherein the oleaginous organism undergoes at least two separate cycles of mixing and recycling.

5. The method of claim 1, wherein the oleaginous organism is an alga.

6. The method of claim 5, wherein the alga is selected from the group consisting of:
Bacillariophyceae strains, Chlorophyceae, Cyanophyceae, Xanthophyceae, Chrysophyceae, Chlorella, Crypthecodinium, Schizocytrium, Nannochloropsis, Ulkenia, Cyclotella, Navicula, Nitzschia, Cyclotella, Phaeodactylum, and Thaustochytrids.

7. The method of claim 1, wherein the oleaginous organism is an oleaginous yeast.

8. The method in claim 7, wherein the yeast is selected from the group consisting of the Rhodotorula, Saccharomyces, and Apiotrichum strains.

9. The method of claim 1, wherein the oleaginous organism is an oleaginous fungus.

10. The method in claim 9 wherein the fungus comprises a Mortierella strain.

11. The method of claim 1, wherein the solvent is selected from the group consisting of C4-C16 hydrocarbons.

12. The method of claim 1, wherein the solvent is selected from the group consisting of C10-C16 hydrocarbons.

13. The method of claim 1, wherein the oleaginous organism is genetically engineered to enhance lipid production.

14. The method of claim 1, wherein the oleaginous organism is concentrated prior to the mixing step.

15. The method of claim 1, wherein sonication is used during at least a portion of the mixing step.

16. The method of claim 15, wherein the sonication is performed at a frequency between 20 kHz and 1 MHz.

17. The method of claim 15, wherein the sonication is performed at a frequency between 20 kHz and 100 kHz.

18. The method of claim 15 wherein the sonication is performed at a frequency of 40 kHz.

19. The method of claim 1, wherein the mixing step is facilitated with at least one of sonication and mechanical mixing.

20. A method for oil extraction from an oleaginous alga, comprising:
mixing at least a portion of a culture containing the alga with a solvent that extracts oil from the alga to obtain a solvent-alga mixture;
directing the solvent-alga mixture into a partitioning chamber to obtain an extracted aqueous fraction containing a viable extracted alga and a solvent-oil fraction; and
recirculating the viable extracted alga into a culturing system.

21. A method for oil extraction from a photosynthetic oleaginous organism, comprising:
mixing at least a portion of a culture containing the photosynthetic oleaginous organism with a solvent that extracts oil from the organism to obtain a solvent-organism mixture;
directing the solvent-organism mixture into a partitioning chamber to obtain an extracted aqueous fraction containing a viable extracted organism and a solvent-oil fraction; and
recirculating the viable extracted organism into a culturing system.

22. The method of claim 21, wherein the method further comprises the step of:
providing a wavelength-shifting dye, the dye adapted to increase the quantity of usable photons available to the photosynthetic alga in the culture system.

23. The method of claim 22, wherein the wavelength-shifting dye is incorporated into particles.

24. The method of claim 22, wherein the wavelength-shifting dye is incorporated into a film.

25. The method of claim 21, wherein said method further comprises the step of:
providing a Fresnel lens adapted to increase the quantity of photons available to the photosynthetic alga when a light source is received at oblique angles.

26. The method of claim 25. wherein a wavelength-shifting dye is incorporated into the Fresnel lens.

27. The method of claim 21, wherein the method further comprises the step of:
distilling the solvent-oil fraction to obtain a usable oil.

28. The method of claim 1, wherein the method is continuous.

29. An apparatus for carrying out the method of claim 1.

30. The method of claim 21, wherein the method is continuous.

31. An apparatus for carrying out the method of claim 21.
